(19) United States
(12) Patent Application Publication
(10) Pub. No.: US 2021/0282700 A1
(43) Pub. Date: Sep. 16, 2021

(54) SEIZURE CHARACTERIZATION WITH MAGNETIC RESONANCE IMAGING (MRI) FUSED WITH AN ELECTROENCEPHALOGRAPHY (EEG) MODEL

(71) Applicants: KONINKLIJKE PHILIPS N.V., EINDHOVEN (NL); PHOENIX CHILDREN'S HOSPTIAL, GLENDALE, AZ (US)

(72) Inventors: Lyubomir Georgiev ZAGORCHEV, BURLINGTON, MA (US); Fabian WENZEL, HAMBURG (DE); Carsten MEYER, HAMBURG (DE); Martin BERGTHOLDT, HAMBURG (DE); Houchun HU, GLENDALE, AZ (US); Jeffrey MILLER, GLENDALE, AZ (US)

(21) Appl. No.: 16/492,179

(22) PCT Filed: Mar. 1, 2018
(86) PCT No.: PCT/EP2018/055004

§ 371 Date: Sep. 9, 2019

Related U.S. Application Data
(60) Provisional application No. 62/469,585, filed on Mar. 10, 2017.

(51) Int. Cl. A61B 5/00; A61B 5/055; A61B 5/369; A61B 5/291; G06T 7/11

(52) U.S. Cl. CPC A61B 5/4094; A61B 5/055; A61B 5/369; G06T 2207/10088; G06T 7/11; G06T 2207/30016; A61B 5/291

(57) ABSTRACT

A seizure characterization method includes correlating locations of electrodes placed around a brain and used to produce sequential electroencephalography (EEG) signals with a three-dimensional anatomical brain model derived from magnetic resonance imaging (MRI). The sequential EEG signals are modelled from the electrodes placed around the brain in three dimensions using cortical and sub-cortical brain regions included in the brain model to define constraints for the numerical solution. Amounts of the sequential EEG signals are quantified in three dimensions relative to the brain regions included in the brain model. The method also includes establishing, based on the quantifying, at least one propagation pattern of the sequential EEG signals in time relative to the brain regions in the brain model.

### BACKGROUND

Epilepsy is a global health concern for humans, characterized by repetitive seizures that are caused by sudden, uncontrolled and intense cerebral electrical discharges originating from specific regions in the brain. In the United States alone, epilepsy affects almost 3 million individuals. By age 75, 3% of the population will develop epilepsy, and 10% of the population will have had at least one seizure. Qualitative magnetic resonance imaging (MRI) is routinely used in clinical assessments to provide three-dimensional detail and high spatial resolution for images of a brain. However, qualitative MRI is non-revealing for many subjects with epileptic seizures.

In subjects with medically intractable epilepsy, if a seizure focus can be localized, surgery to remove the local seizure focus can be curative. When the seizure source cannot otherwise be localized, for example using the qualitative MRI, electroencephalography (EEG) can be acquired. The EEG signal provides important information about electrical discharges in the brain but lacks the three-dimensional detail and high spatial resolution of conventional MRI.

The EEG electrodes are placed on the head to record action potentials such as activation of brain regions. Clusters of the recorded action potentials can be plotted in two dimensions as brain network activation maps, but two-dimensional brain network activation maps do not provide spatial information related to disease-specific anatomy. That is, EEG can be used to effectively monitor electrical activity of seizures, but lacks disease-specific anatomical (spatial) information for the subjects.

Currently, EEG can be combined with multi-modal data sets using advanced commercial software platforms. Multi-modal data sets include, for example, T1. An example of an advanced commercial software platform for combining EEG with such multi-modal data sets is CURRY, as described online at compumedicsneuroscan.com/curry-epilepsy-evaluation. CURRY provides a common framework for spatial localization of an EEG signal in a three-dimensional space. However, CURRY and others do not address, for example, EEG propagation, or three-dimensional EEG in the context of specific brain regions, let alone three-dimensional EEG propagation in the context of specific brain regions. Rather, the role of such advanced commercial software platforms is limited to identifying EEG peaks in the three-dimensional space that may or may not be overlaid with an MRI.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a view of a process for seizure characterization with MRI fused with an EEG model, in accordance with a representative embodiment of the present disclosure.

FIG. 2 is a view of another process for seizure characterization with MRI fused with an EEG model, in accordance with a representative embodiment of the present disclosure.

FIG. 3 is a view of an MRI system for seizure characterization with MRI fused with an EEG model, in accordance with a representative embodiment of the present disclosure.

FIG. 4A is a view of EEG electrodes places around a brain for seizure characterization with MRI fused with an EEG model, in accordance with a representative embodiment of the present disclosure.

FIG. 4B is a view of three-dimensional clustering of action potentials identified based on the use of the EEG electrodes in FIG. 4A, in accordance with a representative embodiment of the present disclosure.

FIG. 5 is a view of cortical and sub-cortical tissue classes in a segmented MRI volume, in accordance with a representative embodiment of the present disclosure.

FIG. 6 is a view of a generalized computer used to implement seizure characterization with MRI fused with an EEG model, in accordance with a representative embodiment of the present disclosure.

FIG. 7 is a view of a procedural timeline and data flow for seizure characterization with MRI fused with an EEG model, in accordance with a representative embodiment of the present disclosure.

FIG. 8 is a view of seizure propagation paths through a head/brain of a subject as modeled using seizure characterization with MRI fused with an EEG model, in accordance with a representative embodiment of the present disclosure.

FIG. 9 is a view of data sets from different propagation paths used in the seizure characterization with MRI fused with an EEG model, in accordance with a representative embodiment of the present disclosure.

### DETAILED DESCRIPTION

In the following detailed description, for purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only, and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In view of the foregoing, the present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

The present disclosure describes a method for combining high resolution structural MRI data with a three-dimensional EEG-based model of brain activation in the context of epileptic seizures. The combined strengths of EEG and MRI help pinpoint seizure onset zones and propagation of EEG signals in three dimensions over time with respect to specific brain regions. The propagation of EEG signals over time can be identified using sequential signal measurement, which in turn can be used to produce and output a sequential display of images (e.g., still image or a video) of the propagation over time. Furthermore, while electrical impulses in the brain can be monitored with EEG, the fusion described herein provides an ability to select a specific isolated brain region and track EEG activity within that specific brain region quantitatively during interventions and and/or during follow-up visits.

From a clinical perspective, the combined ability to pinpoint onset zones and track propagation patterns assists in revealing different patterns of epileptic seizures that can be correlated to symptoms and outcomes. Additionally, the combined pinpointing of onset zones and tracking of propagation patterns can be used as disease biomarkers to differentiate epilepsy sub-types.

Furthermore, the ability to pinpoint the seizure onset zones/location and track propagation helps surgeons limit resection/surgery to mainly the area of seizure onset, and limit the size of removed brain tissue. For example, if the propagation is shown to go across the corpus callosum to the other side of the brain, the surgeons can cut only the corpus callosum to prevent a seizure from affecting the other side of the brain. In other words, the propagation of EEG signals, when linked to anatomy of the specific brain regions, can be used to minimize invasive resections and optimize surgical interventions.

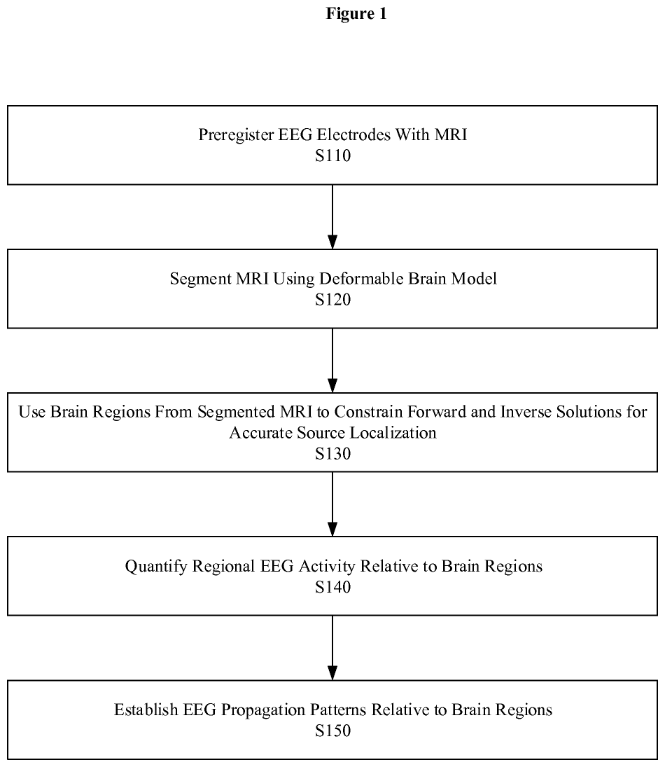

FIG. 1 is a view of a process for seizure characterization with MRI fused with an EEG model, in accordance with a representative embodiment of the present disclosure. FIG. 1 is a high-level overview of processes described herein, starting with preregistration of EEG electrodes with MRI starting at S110. For example, the preregistration can be performed by correlating actual, expected, or intended locations of EEG electrodes with MRI data from the same space as the space measured by the EEG electrodes. In every embodiment described herein, the space includes a brain of a human subject. The underlying correlating may include, for example, correlating the coordinates in a three-dimensional space that includes a brain model created based on MRI with coordinates in a three-dimensional space that includes a brain model created based on EEG signals, so as to ensure that a propagation pattern of the EEG signals is set in a space with comparable coordinates. As explained herein, this allows the propagation pattern of the EEG signals to be, for example, displayed together with a segmented three-dimensional volume of the actual brain regions of the same brain as is subject to the EEG.

In specific examples, the preregistration at S110 can be performed by acquiring the magnetic resonance scan either with compatible EEG electrodes in place or with attached fiducial markers identifying the expected or intended location of EEG electrodes. The EEG electrodes are then later used to record action potentials such as activation of brain regions. The EEG electrodes are usually placed on the head of the human subject, and then action potentials are recorded and then clustered in three dimensions.

At S120, the MRI is segmented using a deformable brain model. The segmentation at S120 is performed by adapting a three-dimensional shape-constrained deformable brain model to structural MRI data from the subject. Production of such a three-dimensional brain model and segmentation of a brain scan are described in, for example, U.S. Patent Application Publication No. 2015/0146951 to ZAGORCHEV et al., published on May 28, 2015, the entire contents of which are incorporated by reference herein.

In more detail, the three-dimensional brain model is segmented at S120 into multiple different brain regions. For the sake of simplicity in the description, locations on a two-dimensional plane can be characterized using, for example, X and Y coordinates, or two sets of alphabetical and/or numeric labels. Locations in a three-dimensional object such as a model can be characterized using X, Y and Z coordinates, or three sets of alphabetical and/or numeric labels. For the brain model segmented at S120, labels can be provided to identify differentiated brain regions, such as brain regions that would be differentiable to one familiar with brain anatomy. To be very clear, the brain regions that can be localized using the fusion described herein are both cortical and sub-cortical brain regions, and may include other regions such as the cerebellum and/or brainstem.

At S130, the brain regions from the segmented MRI obtained at S120 are used to constrain forward and inverse solutions for accurate EEG source localization. In mathematics, a constraint is a condition of an optimization problem that the solution must satisfy. The set of candidate solutions that satisfy all constraints is the feasible set. Furthermore, the solution is defined on a certain geometry with a set of conditions defined on its boundary. Here, in detail, the segmented brain regions from the three-dimensional brain model from the MRI are used to define that geometry as well as the boundary conditions necessary for the forward and inverse solutions to constrain the (brain) space in which EEG signals read by the EEG electrodes preregistered at S110 are allowed to propagate. That is, given the quantification described below with respect to S140, constraints are placed on the boundaries of anatomical structures as defined in the brain model to ensure accurate source localization by quantifying the EEG signal measured on the surface of the brain. In other words, the MRI is used to extract the geometry of brain regions in order to define a detailed geometry and boundary conditions necessary for an accurate solution of the forward and inverse problems.

At S140, the EEG is performed using the EEG electrodes in order to measure brain signals, and the measured EEG brain signals are quantified relative to the segmented MRI brain regions set at S130. The quantifying may be performed by, for example, measuring levels or intensity of the EEG signals at each of a series of consecutive points in time, and then isolating the highest levels and intensities, as well as the locations of the highest levels and intensities, at each of the points in time using the constrained solution of the propagation model. In another alternative embodiment, the average signal within a brain region is measured.

An example of segmentation is shape-constrained deformable segmentation developed by Philips Research, headquartered in Eindhoven in the Netherlands. Shape-constrained deformable segmentation is rapid and fully automatic, and can be applied to three dimensional MRI scans. Shape-constrained deformable segmentation is described in the above-noted U.S. Patent Application Publication No. 2015/0146951. The shape-constrained deformable segmentation can be performed rapidly and automatically on MRI data once the MRI is performed, and the resultant segmented three-dimensional model is adapted specifically to the patient anatomy. When adapted to the subject's scan, the geometry of the EEG model provides a very detailed volumetric mesh that can be tessellated (distributed into objects of equal dimensions) into three-dimensional spatial elements. The propagation of EEG signals is governed by a partial differential equation solved in time over the spatial elements. The solution identifies the source of the EEG signals in the context of the geometry extracted from the segmentation of the structural MRI. Specifically, the solution identifies the seizure onset zone and the propagation of the EEG signal in time as related to specific brain regions.

At S150, propagation patterns are established relative to the brain regions. Specifically, the movement of the isolated levels and intensities over time are used to produce a propagation pattern of the highest EEG brain measurements as they vary in (travel through) the brain regions. As explained herein, these measurements may specifically show the path, timing, and relative effect of a seizure as the seizure induces the brain activity in the different brain regions. The propagation can then be recorded, displayed, reproduced, and even compared with different propagations resulting from seizures suffered by the same subject or other subjects. Of course, since a propagation can be recorded the propagation can also be reproduced, including visually.

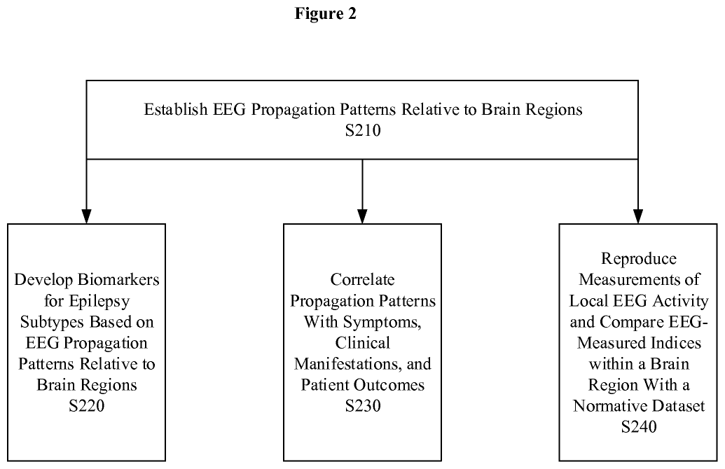

FIG. 2 is a view of another process for seizure characterization with MRI fused with an EEG model, in accordance with a representative embodiment of the present disclosure. In FIG. 2, the EEG propagation patterns are established relative to brain regions/anatomies at S210. The EEG propagation patterns established at S210 are established by the process described in FIG. 1. The EEG propagation patterns may be established multiple times for a single subject over a period of days, weeks, months and even years. For example, a subject may be sent home with a wearable helmet or similar apparatus with the EEG electrodes in fixed locations relative to one another. The subject may then wear the wearable helmet or similar apparatus to ensure that brain activity is measured when different seizures occur.

Alternatively, or additionally, the EEG propagation patterns may be established one or more times for multiple different subjects over a period of days, weeks, months or even years. The EEG propagation patterns established at S210 can be collected from different sources, different locations, different medical providers, different medical facilities, and even in different countries.

At S220, biomarkers are developed based on analysis of the EEG propagation patterns established at S210. As used herein, the term biomarker means a measurable indicator of a biological state or condition. That is, it may be found that multiple subjects with similar EEG propagation paths suffer from the same subtype of epilepsy. At S220, the similarities between propagation paths are correlated as biomarkers. Of course, the biomarkers may be correlated with other characteristics besides propagation paths, such as subject demographics (e.g., age, race, gender).

At S230, the propagation patterns are correlated with symptoms, clinical manifestations, and subject outcomes. That is, propagation patterns of each subject can be correlated with other health symptoms of the subject affected by the seizures. The propagation patterns can be correlated with clinical manifestations that evidence symptoms to a trained observer (e.g., doctor or researcher) or to the subject who exhibits the symptoms. Finally, the propagation patterns can be correlated with subject outcomes, such as resolutions based on successful interventions (e.g., surgery or medication).

A benefit of the correlation at S230 is that once propagation patterns can be correlated with symptoms, clinical manifestations and subject outcomes, a propagation pattern newly identified for a subject can be used to assist the subject. Similarly, a subject exhibiting a particular symptom or clinical manifestation may be subject to the seizure characterization with MRI fused with an EEG model described herein, with the expectation that the concepts described herein may confirm a diagnosis and treatment plan.

The propagation of the EEG signal can be modelled in three dimensions using a finite difference method, a finite element method, and/or a boundary elements method. All three will essentially start with the EEG signal detected at the EEG electrodes and then back-propagate it within the tessellated spatial grid or elements representing the brain based on the MRI.

The quantified EEG activity can be indexed, so that comparative values are assigned for different seizures and different subjects. Using indexed values, normative data sets can be developed for use in comparing different seizures for a single subject or for multiple different subjects.

At S240, measurements of the local EEG activity can be reproduced and compared with EEG-measured indices within a brain region with a normative dataset. Normative data is data that characterizes a baseline for a reference population. At S240, the local EEG activity for a particular brain region or regions can be compared with average, median, typical or other expected EEG activity. As noted previously, the brain regions subject to the fusion described herein are not just cortical brain regions, but also include subcortical brain regions. The normative data may be based on EEG measurements from the same subject when the subject is not suffering from a seizure, and/or may be EEG measurements from other subjects when they are not suffering from a seizure, and/or may be EEG measurements from the same or other subjects specifically when they are suffering from a seizure. In this way, EEG measurements during a particular seizure can be compared with expected, typical EEG measurements from the same or other subjects when they are or are not suffering from seizures.

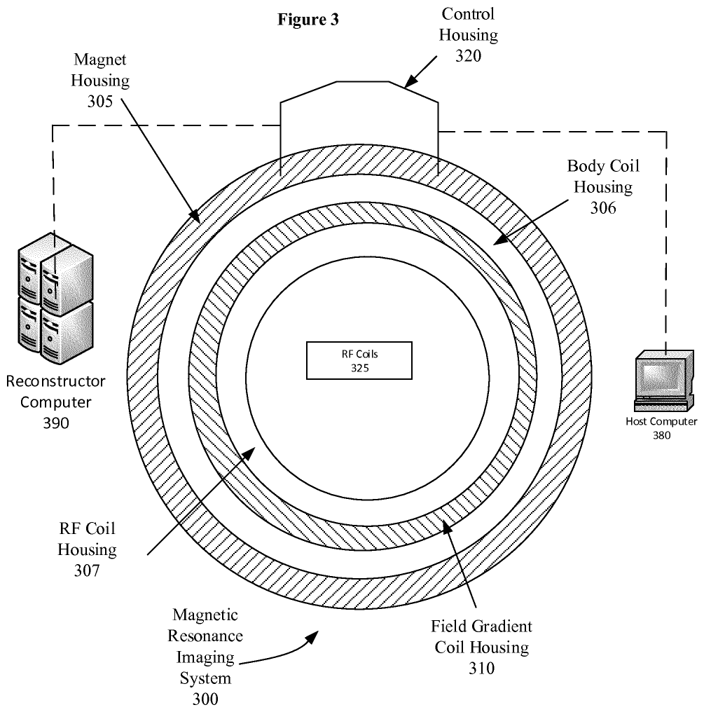

FIG. 3 is a view of an MRI system for seizure characterization with MRI fused with an EEG model, in accordance with a representative embodiment of the present disclosure. In FIG. 3, a magnet housing 305 is designated with a hatch pattern as an outer structure of a MRI system 300. A body coil housing 306 is immediately interior to the magnet housing 305. A field gradient coil housing 310 is immediately interior to the body coil housing 306. A radio frequency (RF) coil housing 307 is immediately interior to the field gradient coil housing 310. A control housing 320 is provided on the magnet housing 305 to house, e.g., external circuitry such as a transceiver.

In FIG. 3, radio frequency coils 325 are body coils placed on the body of the subject/subject who is subjected to the MRI scan. The radio frequency signals are emitted from the MRI system 300 to excite the hydrogen atoms, and the hydrogen atoms emanate a weak radio frequency signal. The radio frequency signals from the hydrogen atoms are the signals with the intensity that is represented in the data created by the MRI system 300. In the fusion described herein, the MRI scan may be, for example, a T1 scan.

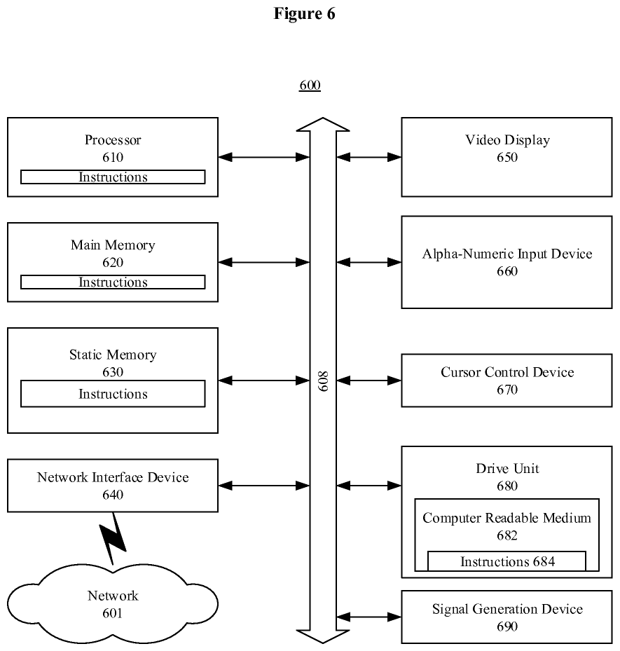

In FIG. 3, two computers included with the MRI system 300 include the reconstructor computer 390 and the host computer 380. The host computer 380 interfaces with an operator of the MRI system 300 to control the MRI system 300 and to collect the images. The reconstructor computer 390 is a “background” computer that acts as a gatekeeper for data flow and computes a three dimensional image from the recorded data. The reconstructor computer 390 does not interact with the operator. Although not shown in FIG. 3, data may also be taken offline so that analysis may be performed on a, for example desktop, computer using software that may be proprietary to the manufacturer of the MRI system 300. FIG. 6 shows a general computer system that may partially or fully be used to implement the reconstructor computer 390 and host computer 380, as well as any other computer or computing device that performs part or all of methods described herein.

As described with respect to FIG. 1, MRI may be performed by MRI system 300 with EEG electrodes or fiducial markers on the head of a subject. Additionally, the MRI information obtained from the MRI of the subject's brain can be modeled based on a preexisting deformable model in three dimensions and then segmented to reflect differentiable brain regions of the subject. The subject may undergo the MRI as few as one time, and the resultant segmented MRI of the brain can be used repeatedly for each subsequent reading of EEG signals during different seizures. In other words, a subject may only need to undergo MRI once in order to obtain the benefits of seizure characterization with MRI fused with an EEG model. Longitudinal follow-up studies can be performed with only EEG, registered to the baseline magnetic resonance scan for accurate spatial localization. As an example, a home-based EEG monitoring application can be used to track the progression of disease/treatment, or just to monitor the condition of a subject. The baseline magnetic resonance scan can be acquired once in a clinical setting and the EEG signals can be acquired repeatedly at home and mapped to the MRI baseline remotely. Each time, the solution method can be applied to start with the EEG signal detected at EEG electrodes on the surface of the subject's brain (head) and then propagate the EEG signal readings back within the tessellated spatial elements to the source. The propagation meets the requirements of a partial differential equation solved in time over the tessellated spatial elements, and the solution identifies the source in the geometry from the extracted segmentation.

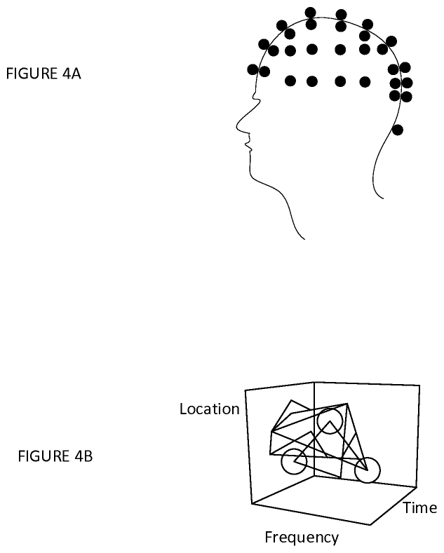

FIG. 4A is a view of EEG electrodes placed around a brain for seizure characterization with MRI fused with an EEG model, in accordance with a representative embodiment of the present disclosure. In FIG. 4A, the EEG electrodes are placed at intended or expected positions around a subject's head (brain). The EEG electrodes may be fixed in place relative to a wearable device to which all EEG electrodes are attached.

FIG. 4B is a view of three-dimensional clustering of action potentials within anatomical brain regions identified based on the use of the EEG electrodes in FIG. 4A. In FIG. 4B, clusters of signals with intensities above, for example, a predetermined threshold, are identified in the three-dimensional space shown in FIG. 4B. The clusters are shown for identified signals with similar locations, frequencies of occurrence, and times. The locations are locations in the brain as identified by the relative location of the particular EEG electrode(s) that detect the signal. The frequencies are the number of times the signal is measured. The time is the relative time in the time sequence during which the EEG electrodes are used to measure the EEG signals in a segment, such as a segment of time when a seizure is occurring. The clusters are indicated by the circles places in three locations in FIG. 4B.

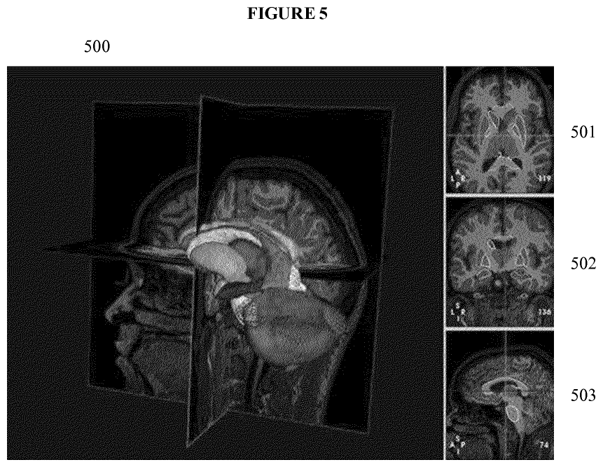

FIG. 5 is a view of cortical tissue classes and sub-cortical structures in a segmented MRI volume, in accordance with a representative embodiment of the present disclosure. As shown in FIG. 5, the head of the subject is divided into eight (8) regions. In FIG. 5, the 8 regions are defined by the three orthogonal planes; however, the location of the orthogonal planes is defined by a point in a three-dimensional volume (X, Y, Z). An operator can click on a different point on any of the planes to change the location of the orthogonal planes. The regions can be color-coded into several or even many different colors. It should be noted that the brain regions are not specifically equivalent volumes or shapes, and reflect actual brain tissue characteristics rather than strict geometric characteristics. In FIG. 5, examples of the tissue classes can be grey matter, white matter, and cerebrospinal fluid in 3-D view of the multi-planar representation showing the brain model. The brain regions in FIG. 5 include both cortical and subcortical brain regions, and the propagation of EEG signals described herein is traced through both the cortical and subcortical brain regions.

In FIG. 5, four separate images are shown together for the same three-dimensional cerebral cortex 500 from a segmented T-1 weighted volume. In the left image, segmentation is shown to include visually bisecting the three-dimensional cerebral cortex with three planes, i.e., an axial, coronal, and sagittal plane from top to bottom on the right.

In FIG. 5, the three 2-dimensional images 501, 502, 503 to the right are a 2-dimensional axial cross-section image 501, a 2-dimensional coronal cross section image 502, and a 2-dimensional sagittal cross section image 503. All three of the 2-dimensional images 501, 502, 503 are projections onto the three bisecting planes shown in the three-dimensional image to the left.

FIG. 6 is an illustrative embodiment of a general computer system, on which a method of seizure characterization with MRI fused with an EEG model can be implemented, and which is shown and is designated 600. The computer system 600 can include a set of instructions that can be executed to cause the computer system 600 to perform any one or more of the methods or computer based functions disclosed herein. The computer system 600 may operate as a standalone device or may be connected, for example, using a network 601, to other computer systems or peripheral devices.

In a networked deployment, the computer system 600 may operate in the capacity of a server or as a client user computer in a server-client user network environment, or as a peer computer system in a peer-to-peer (or distributed) network environment. The computer system 600 can also be implemented as or incorporated into various devices, such as a stationary computer, a mobile computer, a personal computer (PC), a laptop computer, a tablet computer, a wireless smart phone, a communications device, a control system, a web appliance, a reconstructor computer, a host computer, or any other machine capable of executing a set of instructions (sequential or otherwise) that specify actions to be taken by that machine. The computer system 600 can be incorporated as or in a particular device that in turn is in an integrated system that includes additional devices. In a particular embodiment, the computer system 600 can be implemented using electronic devices that provide video and/or data communication. Further, while a single computer system 600 is illustrated, the term “system” shall also be taken to include any collection of systems or sub-systems that individually or jointly execute a set, or multiple sets, of instructions to perform one or more computer functions.

As illustrated in FIG. 6, the computer system 600 includes a processor 610. A processor for a computer system 600 is tangible and non-transitory. As used herein, the term “non-transitory” is to be interpreted not as an eternal characteristic of a state, but as a characteristic of a state that will last for a period of time. The term “non-transitory” specifically disavows fleeting characteristics such as characteristics of a particular carrier wave or signal or other forms that exist only transitorily in any place at any time. A processor is an article of manufacture and/or a machine component. A processor for a computer system 600 is configured to execute software instructions in order to perform functions as described in the various embodiments herein. A processor for a computer system 600 may be a general-purpose processor or may be part of an application specific integrated circuit (ASIC). A processor for a computer system 600 may also be a microprocessor, a microcomputer, a processor chip, a controller, a microcontroller, a digital signal processor (DSP), a state machine, or a programmable logic device. A processor for a computer system 600 may also be a logical circuit, including a programmable gate array (PGA) such as a field programmable gate array (FPGA), or another type of circuit that includes discrete gate and/or transistor logic. A processor for a computer system 600 may be a central processing unit (CPU), a graphics processing unit (GPU), or both. Additionally, any processor described herein may include multiple processors, parallel processors, or both. Multiple processors may be included in, or coupled to, a single device or multiple devices.

Moreover, the computer system 600 includes a main memory 620 and a static memory 630 that can communicate with each other via a bus 608. Memories described herein are tangible storage mediums that can store data and executable instructions, and are non-transitory during the time instructions are stored therein. As used herein, the term “non-transitory” is to be interpreted not as an eternal characteristic of a state, but as a characteristic of a state that will last for a period of time. The term “non-transitory” specifically disavows fleeting characteristics such as characteristics of a particular carrier wave or signal or other forms that exist only transitorily in any place at any time. A memory described herein is an article of manufacture and/or machine component. Memories described herein are computer-readable mediums from which data and executable instructions can be read by a computer. Memories as described herein may be random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. Memories may be volatile or non-volatile, secure and/or encrypted, unsecure and/or unencrypted.

As shown, the computer system 600 may further include a video display unit 650, such as a liquid crystal display (LCD), an organic light emitting diode (OLED), a flat panel display, a solid-state display, or a cathode ray tube (CRT). Additionally, the computer system 600 may include an input device 660, such as a keyboard/virtual keyboard or touch-sensitive input screen or speech input with speech recognition, and a cursor control device 670, such as a mouse or touch-sensitive input screen or pad. The computer system 600 can also include a disk drive unit 680, a signal generation device 690, such as a speaker or remote control, and a network interface device 640.

In a particular embodiment, as depicted in FIG. 6, the disk drive unit 680 may include a computer-readable medium 682 in which one or more sets of instructions 684, e.g. software, can be embedded. Sets of instructions 684 can be read from the computer-readable medium 682. Further, the instructions 684, when executed by a processor, can be used to perform one or more of the methods and processes as described herein. In a particular embodiment, the instructions 684 may reside completely, or at least partially, within the main memory 620, the static memory 630, and/or within the processor 610 during execution by the computer system 600.

In an alternative embodiment, dedicated hardware implementations, such as application-specific integrated circuits (ASICs), programmable logic arrays and other hardware components, can be constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing can be constructed to implement one or more of the methods or functionality as described herein, and a processor described herein may be used to support a virtual processing environment.

The present disclosure contemplates a computer-readable medium 682 that includes instructions 684 or receives and executes instructions 684 responsive to a propagated signal; so that a device connected to a network 601 can communicate voice, video or data over the network 601. Further, the instructions 684 may be transmitted or received over the network 601 via the network interface device 640.

Notably, computers in or around the immediate vicinity of a MRI system 300, may vary from typical computers to ensure they do not interfere with the operation of the MRI system 300. For example, a computer system 600 may be modified to ensure that it emits no or negligible magnetic or radio frequency transmissions. However, as noted herein, a MRI session need only be performed once for a variety of the embodiments described herein. The sequential EEG signals can be repeatedly acquired remote from any MRI system 300, and then applied to the same, single, existing brain model derived from MRI of the subject's brain.

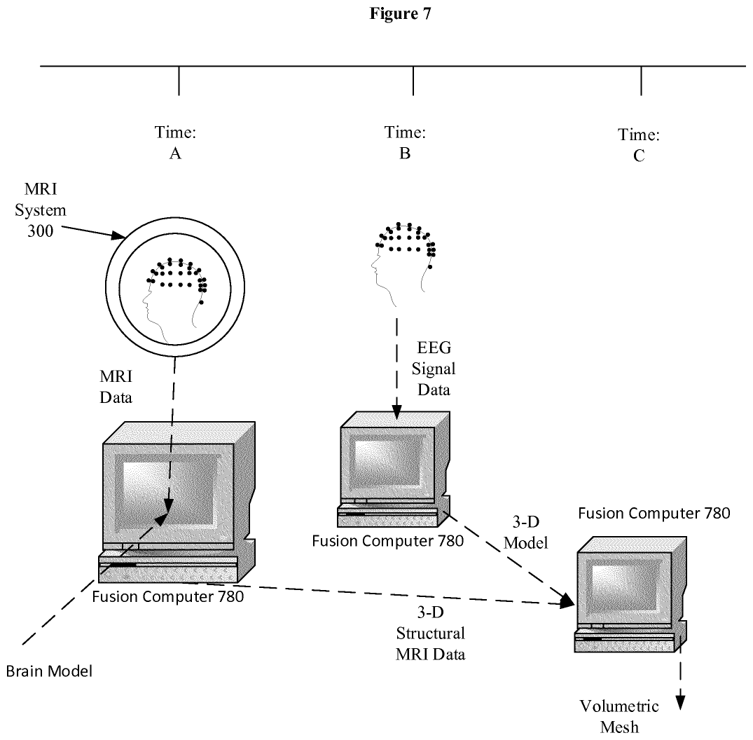

FIG. 7 is a view of a procedural timeline and data flow for seizure characterization with MRI fused with an EEG model, in accordance with a representative embodiment of the present disclosure. In FIG. 7, a timeline at the top shows Times A, B and C. The first time, Time A, is when a MRI system such as MRI System 300 is used to perform MRI on a subject. The MRI can be performed with EEG electrodes or fiducial markers in place in order to correlate locations of subsequent EEG signals with the segmented three-dimensional model resulting from the MRI. A preexisting brain model is applied to the MRI data to obtain the segmented three-dimensional model specific to the patient. The results of the MRI and subsequent segmenting at Time A are fed to a fusion computer 780 as three dimensional structural MRI data. More particularly, the results from applying the brain model to the MRI data for the patient provide the geometry for accurate solutions of the forward and inverse problems required to generate a three-dimensional EEG model as described next for Time B.

At the second time, Time B, EEG signals are collected from EEG electrodes placed around a subject's brain. The EEG signals are used to generate a three-dimensional model of the data, such as by the quantifying described already. The EEG signal data is transformed to the three-dimensional model by the fusion computer 780.

At the third time, Time C, the structural MRI data from the segmenting at Time A is fused with the three-dimensional model of EEG data by the fusion computer 780. The result is a volumetric mesh from the segmented three-dimensional structural MRI with the propagation path from the three-dimensional EEG signal superimposed therein. The propagation path corresponds to quantified EEG signals at specific locations from/in the segmented three-dimensional structural MRI showing the different brain regions.

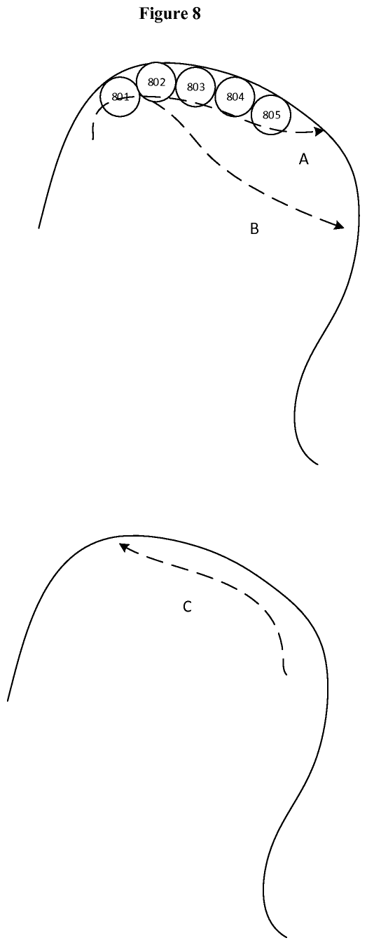

FIG. 8 is a view of seizure propagation paths through a head/brain of a subject as modeled using seizure characterization with MRI fused with an EEG model, in accordance with a representative embodiment of the present disclosure. FIG. 8 shows three different propagation paths A, B and C in outlines of a head of a subject. The main point to be derived from FIG. 8 is that a propagation path can start from different regions of the brain, and can travel through different regions for different seizures. This is true whether the seizures A, B and C are all incurred by one subject, or three different subjects. In FIG. 8, five (5) arbitrary regions of the brain are shown as identical circles designated as 801, 802, 803, 804 and 805. Of course, essentially no brain region is accurately represented as a circle, and the use of circles in FIG. 8 is used to show that propagation can be shown as a path superimposed on actual brain regions derived from an MRI.

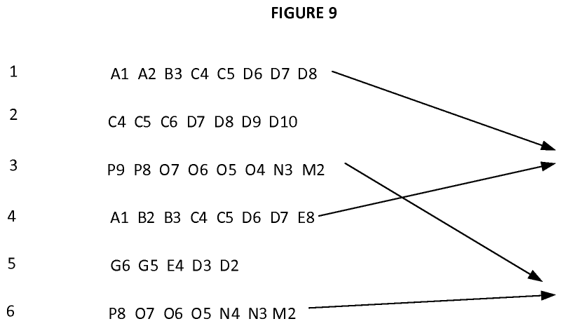

FIG. 9 is a view of data sets from different propagation paths used in the seizure characterization with MRI fused with an EEG model, in accordance with a representative embodiment of the present disclosure. In FIG. 9, six (6) different data sets are shown as alphanumeric combinations. The first data point in each data set corresponds to a different brain region. As an example, the different letters A, B, C, D, P, O, N, M, G etc. may correspond to different distinguishable depth levels of the brain, and the numbers may correspond to delineations from, for example, front to back or left side to right side.

In an analysis of the data sets in FIG. 9, similar data sets may be identified and grouped together. The similarities may be found based on the origin (first data point) in sets, the absolute number of identical brain regions affected, similarities in the path direction, or any other similarities that may be found from a set of alphanumeric data. Each data set in a group will be associated with health information, subject information and so on, to check for similarities in subjects, symptoms, clinical manifestations and so on. In this way, the fusion described herein is used to identify commonalities between different seizures and different subjects with similar patterns of propagations and, for example, symptoms. Mappings from data to categories can also be found by machine learning and/or data mining techniques. This is true insofar as epileptic seizures are common such that massive amount of data can be obtained from monitoring subjects who suffer from such seizures.

An example of the benefits that can result from the identification of propagation patterns is an ability to establish successful resolutions for different subtypes of epilepsy, such as by correlating a specific propagation pattern with limited type of surgical resection to pinpoint mainly the area of seizure onset, and to limit the size/amount of removed brain tissue. In other words, the propagation of EEG signal, when linked to brain regions, can be used for minimally invasive resections and optimization of surgical interventions.

Additionally, the recording of propagations from quantified EEG signals relative to an MRI volume can be used to evaluate the success or failure of treatments. For example, a benefit might be obtained if the relative amount of detected EEG signals in regions is reduced, or the length of propagation is reduced. Similarly, a particular type of treatment can be deemed effective when it stops seizures in subjects that exhibit a particular type of propagation, even if other subjects with other propagations do not benefit.

Accordingly, seizure characterization with MRI fused with an EEG model enables tracking of EEG activity within a specific brain region to identify seizure onset zones. In turn, the tracking of EEG activity can result in enhancements for surgical planning/interventions, and recovery monitoring. For example, accurate localization of seizure origins and the consequent propagation patterns can reveal specific characteristics that can be correlated with disease symptoms and outcomes.

Although seizure characterization with MRI fused with an EEG model has been described with reference to several exemplary embodiments, it is understood that the words that have been used are words of description and illustration, rather than words of limitation. Changes may be made within the purview of the appended claims, as presently stated and as amended, without departing from the scope and spirit of seizure characterization with MRI fused with an EEG model in its aspects. Although seizure characterization with MRI fused with an EEG model has been described with reference to particular means, materials and embodiments, seizure characterization with MRI fused with an EEG model is not intended to be limited to the particulars disclosed; rather seizure characterization with MRI fused with an EEG model extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

According to an aspect of the present disclosure, a seizure characterization method includes correlating locations of electrodes placed around a brain and used to produce sequential EEG signals with a three-dimensional brain model derived from MRI. The sequential EEG signals from the electrodes placed around the brain are modelled in three dimensions using cortical and sub-cortical brain regions included in the brain model as constraints. Amounts of the sequential EEG signals are quantified in three dimensions relative to the brain regions included in the brain model. The method also includes establishing, based on the quantifying, at least one propagation pattern of the sequential EEG signals in time relative to the brain regions in the brain model.

According to another aspect of the present disclosure, the seizure characterization includes obtaining the sequential EEG signals using the electrodes. The sequential EEG signals are mapped to the brain model to establish at least one propagation pattern.

According to yet another aspect of the present disclosure, the seizure characterization method includes obtaining the sequential EEG signals using the electrodes multiple different times. The sequential EEG signals are mapped to the brain model each different time to establish multiple propagation patterns.

According to still another aspect of the present disclosure, the seizure characterization method includes comparing the propagation pattern with a plurality of propagation patterns relative to brain regions in other brain models. A characteristic common to only a subset of the compared propagation patterns is identified based on the comparing.

According to another aspect of the present disclosure, the seizure characterization method includes visually isolating the propagation pattern.

According to yet another aspect of the present disclosure, the seizure characterization method includes segmenting the brain model into the cortical and sub-cortical brain regions.

According to still another aspect of the present disclosure, the seizure characterization method includes using the brain regions from the brain model to constrain forward and inverse solutions of the propagation pattern relative to the brain regions.

According to another aspect of the present disclosure, the sequential EEG signals are generated based on a seizure passing through the cortical and sub-cortical regions of the brain in three dimensions over time from a source region in the brain.

According to yet another aspect of the present disclosure, the seizure characterization method includes isolating a brain region from which the seizure originates in relation to the brain model.

According to still another aspect of the present disclosure, the seizure characterization method includes isolating one of the brain regions, and tracking sequential EEG signals from the isolated brain region.

According to another aspect of the present disclosure, the modelling is performed using a finite difference method, a finite element method, or a boundary element method

According to yet another aspect of the present disclosure, the modelling method is applied starting with the sequential EEG signals detected at the electrodes around the brain, and back-propagates the detected sequential EEG signals within tessellated spatial elements generated by the segmentation provided by the deformable brain model.

According to still another aspect of the present disclosure, the segmentation comprises shape-constrained deformable segmentation and produces either a volumetric mesh of the brain regions tessellated into spatial elements, or a binary bitmask representing each anatomical brain region.

According to another aspect of the present disclosure, the shape-constrained deformable segmentation is performed automatically by a processor using results of the MRI scan.

According to yet another aspect of the present disclosure, the seizure characterization method includes the segmentation provided by the deformable brain model adapted to a specific subject.

According to still another aspect of the present disclosure, sequential EEG signals are quantified repeatedly for the subject. A propagation pattern is established each time based on the same brain model.

According to an aspect of the present disclosure, a seizure characterization method includes correlating locations of electrodes placed around a brain and used to produce sequential EEG signals with a three-dimensional brain model derived from MRI. The brain model is segmented into cortical and sub-cortical brain regions. The sequential EEG signals from the electrodes placed around the brain are modelled in three dimensions using the segmented cortical and sub-cortical brain regions included in the brain model as constraints. Amounts of the sequential EEG signals are quantified in three dimensions relative to the brain regions included in the brain model. The method also includes establishing, based on the quantifying, at least one propagation pattern of the sequential EEG signals in time relative to the brain regions in the brain model. The sequential EEG signals are generated based on a seizure passing through the cortical and/or sub-cortical regions of the brain in three dimensions over time from a source region in the brain.

According to yet another aspect of the present disclosure, the seizure characterization method includes generating a progression of images showing the propagation pattern in three dimensions. The sequential EEG signals in three dimensions show activity of the brain as the seizure induces the sequential EEG signals.

According to an aspect of the present disclosure, a seizure characterization method includes correlating locations of electrodes placed around a plurality of brains and used to produce sequential EEG signals with three-dimensional brain models derived from MRI. The sequential EEG signals from the electrodes placed around each of the brains are modelled using cortical and sub-cortical brain regions included in the brain models as constraints. Amounts of the sequential EEG signals are quantified in three dimensions relative to the brain regions included in the brain models. The method also includes establishing, based on the quantifying, propagation patterns of the sequential EEG signals in time relative to the brain regions of each of the corresponding brain models. The propagation patterns are compared to identify a commonality among a subset of the propagation patterns.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1. A seizure characterization method, comprising:
correlating locations of electrodes placed around a brain and used to produce electroencephalography (EEG) signals with a three-dimensional brain model derived from magnetic resonance imaging (MRI);
modelling the EEG signals from the electrodes placed around the brain in three dimensions using sub-cortical brain regions included in the brain model to define constraints for a numerical solution;
quantifying amounts of the electrical activity based on the EEG signals in three dimensions relative to the brain regions included in the brain model; and
establishing, based on the quantifying, at least one propagation pattern of the EEG signals in time relative to the brain regions in the brain model.

2. The seizure characterization method of claim 1, further comprising:
obtaining the EEG signals using the electrodes; and
mapping the EEG signals to the brain model to establish the at least one propagation pattern.

3. The seizure characterization method of claim 1, further comprising:
obtaining the EEG signals using the electrodes multiple different times; and
mapping the EEG signals to the brain model each different time to establish multiple propagation patterns.

4. The seizure characterization method of claim 1, further comprising:
comparing the propagation pattern with a plurality of propagation patterns relative to brain regions in other brain models, and
identifying a characteristic common to only a subset of the compared propagation patterns.

5. The seizure characterization method of claim 1, further comprising:
visually isolating the propagation pattern.

6. The seizure characterization method of claim 1, further comprising:
segmenting the brain model into the sub-cortical brain regions of the brain.

7. The seizure characterization method of claim 6, further comprising:
using the brain regions of the brain from the segmented brain model to constrain forward and inverse solutions of the propagation pattern relative to the brain regions of the brain.

8. The seizure characterization method of claim 6,
wherein the EEG signals are generated based on a seizure passing through the sub-cortical regions of the brain in three dimensions over time from a source region in the brain.

9. The seizure characterization method of claim 8, further comprising:
identifying a brain region of the brain from which the seizure originates in relation to the brain model.

10. The seizure characterization method of claim 6, further comprising:
identifying one of the brain regions, and tracking EEG signals from the isolated brain regions.

11. The seizure characterization method of claim 6, further comprising:
wherein the modelling is performed using at least one of a boundary element method, a finite difference method, or a finite element method.

12. The seizure characterization method of claim 11, wherein the boundary element method is applied starting with the EEG signals detected at the electrodes around the brain, and back-propagates the detected EEG signals within tessellated spatial elements within sub-cortical regions generated in the segmentation provided by the brain model.

13. The seizure characterization method of claim 6,
wherein the segmentation produces a volumetric mesh of the brain regions of the brain tessellated into spatial elements.

14. The seizure characterization method of claim 13, wherein the segmentation is performed by a processor using results of the MRI.

15. The seizure characterization method of claim 6, wherein the segmented brain model is specific to a subject.

16. (canceled)

17. A seizure characterization method, comprising:
correlating locations of electrodes placed around a brain and used to produce electroencephalography (EEG) signals with a three-dimensional brain model derived from magnetic resonance imaging (MRI);
segmenting the brain model into cortical and sub-cortical brain regions of the brain;
modelling the EEG signals from the electrodes placed around the brain in three dimensions using the segmented cortical and sub-cortical brain regions included in the brain model to define constraints for a numerical solution;
quantifying amounts of the EEG signals in three dimensions relative to the brain regions included in the brain model; and
establishing, based on the quantifying, at least one propagation pattern of the EEG signals in time relative to the brain regions in the brain model,
wherein the EEG signals are generated based on a seizure passing through the cortical and sub-cortical regions of the brain in three dimensions over time from a source region in the brain.

18. The seizure characterization method of claim 17, further comprising:
generating a progression of images showing the propagation pattern in three dimensions,
wherein the EEG signals in three dimensions show activity of the brain as the seizure induces the EEG signals.

19. (canceled)

20. The seizure characterization method of claim 1, wherein the EEG signals are at least one of continuous or sequential.

21. A seizure characterization method, comprising:
modelling electroencephalography (EEG) signals in three dimensions based on a sub-cortical brain region included in a three dimensional brain model derived from magnetic resonance imaging data of a subject; and
establishing at least one seizure propagation pattern of the EEG signals in time relative to the sub-cortical brain region.
